(19) United States
(12) Patent Application Publication
(10) Pub. No.: US 2018/0344248 A1
(43) Pub. Date: Dec. 6, 2018

(54) CONTRAST REMOVAL SYSTEM

(71) Applicant: BOSTON SCIENTIFIC SCIMED, INC., Maple Grove, MN (US)

(72) Inventors: HONGXIA ZENG, Maple Grove, MN (US); DONGMING HOU, Plymouth, MN (US); HONG CAO, Maple Grove, MN (US); BIN MI, Arden Hills, MN (US)

(73) Assignee: BOSTON SCIENTIFIC SCIMED, INC., Maple Grove, MN (US)

(21) Appl. No.: 15/995,564

(22) Filed: Jun. 1, 2018

Related U.S. Application Data
(60) Provisional application No. 62/549,139, filed on Aug. 23, 2017.
(60) Provisional application No. 62/514,649, filed on Jun. 2, 2017.

(51) Int. Cl. A61B 5/00; A61M 5/00; A61B 5/053; A61M 1/00; A61M 25/10

(52) U.S. Cl. CPC A61B 5/6852; A61M 5/007; A61B 5/0538; A61M 25/04; A61M 25/10; A61B 5/489; A61M 1/0031

(57) ABSTRACT

Medical systems and methods for making and using medical systems are disclosed. An example may include a catheter, a sensor, and a pump configured to remove contrast from a vascular system. The catheter may have a lumen through which the pump may suction contrast. The sensor may be positioned distal of and upstream of a distal end of the aspiration lumen and the pump may initiate suction through the lumen in response to a value sensed by the sensor reaching and/or going beyond a threshold value. The catheter may include an expandable member configured to expand in response to a value sensed by the sensor reaching and/or going beyond a threshold value. The sensor may be supported by a distal extension of the catheter or an elongate member configured to extend through and/or along the catheter to position the sensor distal of the distal end of the aspiration lumen.

### CROSS-REFERENCE TO RELATED APPLICATIONS

This application claims priority under 35 U.S.C. § 119 to U.S. Provisional Application Ser. No. 62/514,649, filed Jun. 2, 2017, the entirety of which is incorporated herein by reference. This application claims priority under 35 U.S.C. § 119 to U.S. Provisional Application Ser. No. 62/549,139, filed Aug. 23, 2017, the entirety of which is incorporated herein by reference.

### TECHNICAL FIELD

The present disclosure pertains to medical devices, and methods for manufacturing and using medical devices. More particularly, the present disclosure pertains to catheter and guidewire devices, methods, and systems, including those with sensing and aspirating capabilities.

### BACKGROUND

A wide variety of medical devices have been developed for medical use, for example, for use in accessing body cavities and interacting with fluids in body cavities. Some of these devices may include guidewires, catheters, pumps, filters, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### BRIEF SUMMARY

This disclosure provides, design, material, manufacturing method, and use alternatives for medical devices and systems. In a first aspect, a catheter system may comprise a catheter, the catheter including one or more lumens, an elongate member advanceable through a lumen of the one or more lumens such that a distal end portion of the elongate member extends distally of a distal end of the catheter, a sensor positioned at a distal end portion of the elongate member and configured to sense a value at a location distal of the distal end of the catheter, and an aspiration pump in communication with an aspiration lumen of the one or more lumens of the catheter, wherein the aspiration pump may initiate aspiration in response to the sensor sensing a value reaches and/or is beyond an aspiration threshold value.

In addition or alternative and in a second aspect, the catheter system may further comprise a controller in communication with the sensor, wherein the controller may be configured to receive the value from the sensor and compare the value to the aspiration threshold value.

In addition or alternative and in a third aspect, the catheter may include an expandable member having a collapsed configuration and an expanded configuration, and the expandable member may be configured to expand to the expanded configuration in response to the value from the sensor reaching and/or going beyond an expansion threshold value.

In addition or alternative and in a fourth aspect, the catheter system may further comprise an inflation pump in communication with the expandable member, wherein the inflation pump may be configured to adjust the expandable member between the collapsed configuration and the expanded configuration in response to a value from the sensor reaching and/or going beyond the expansion threshold value.

In addition or alternative and in a fifth aspect, the catheter system may further comprise a controller in communication with the sensor, wherein the controller may be configured to receive the value from the sensor and compare the value to the expansion threshold value.

In addition or alternative and in a sixth aspect, the controller may be configured to initiate expanding the expandable member to the expanded configuration in response to determining the value reaches and/or is beyond the expansion threshold value.

In addition or alternative and in a seventh aspect, the expansion threshold value is the same as the aspiration threshold value.

In addition or alternative and in an eighth aspect, the controller may be configured to initiate expanding the expandable member to the expanded configuration at a first time and is configured to actuate the aspiration pump at a second time after the first time.

In addition or alternative and in a ninth aspect, the catheter system may further comprise a filter in communication with the aspiration lumen, wherein the filter may receive fluid passing through the aspiration lumen in response to actuation of the aspiration pump.

In addition or alternative and in a tenth aspect, the value sensed by the sensor may include an impedance measure of a fluid at the location distal of the distal end of the catheter.

In addition or alternative and in an eleventh aspect, the value sensed by the sensor may include a temperature measure of a fluid at the location distal of the distal end of the catheter.

In addition or alternative and in a twelfth aspect, the value sensed by the sensor may include a wavelength measure of a fluid at the location distal of the distal end of the catheter.

In addition or alternative and in a thirteenth aspect, the value sensed by the sensor may include one or more of an impedance measure of a fluid at the location distal of the distal end of the catheter, a temperature measure of a fluid at the location distal of the distal end of the catheter, and a wavelength measure of a fluid at the location distal of the distal end of the catheter.

In addition or alternative and in a fourteenth aspect, the catheter may comprise an outer catheter, and an inner catheter extending within a lumen of the outer catheter, wherein the inner catheter comprises the aspiration lumen.

In addition or alternative and in a fifteenth aspect, the catheter comprises an outer catheter having an opening through a side wall defining a lumen of the outer catheter, and an inner catheter extending within the lumen of the outer catheter, wherein the inner catheter is movable relative to the outer catheter to selectively cover the opening. In addition or alternative and in a sixteenth aspect, a contrast removal system for removing contrast from a vascular system may comprise a catheter having an expandable member and an aspiration lumen having a distal end at location distal of the expandable member, a pump in communication with the aspiration lumen, a sensor positionable at a location distal of the distal end of the aspiration lumen, the sensor may be configured to sense values in fluid at the location distal of the distal end of the aspiration lumen, a controller in communication with the pump and the sensor, and wherein the controller may be configured to receive values from the sensor, compare the values to a threshold value, and initiate the pump when a value reaches and/or is beyond the threshold value.

In addition or alternative and in a seventeenth aspect, the controller may be configured to initiate expanding the expandable member from a collapsed configuration to an expanded configuration in response to the value reaching and/or going beyond the threshold value.

In addition or alternative and in an eighteenth aspect, the catheter may comprise an outer catheter, and an inner catheter extending within a lumen of the outer catheter, wherein the inner catheter comprises the aspiration lumen.

In addition or alternative and in a nineteenth aspect, the catheter may comprise an outer catheter having an opening through a side wall defining a lumen of the outer catheter, and an inner catheter extending within the lumen of the outer catheter, wherein the inner catheter is movable relative to the outer catheter to selectively cover the opening.

In addition or alternative and in a twentieth aspect, the system may further comprise an inflation pump, wherein initiating expansion of the expandable member from the collapsed configuration to the expanded configuration may include actuating the inflation pump to supply inflation fluid to the expandable member.

In addition or alternative and in a twenty first aspect, the system may further comprise a filter in fluid communication with the aspiration lumen.

In addition or alternative and in a twenty second aspect, the catheter may comprise an extension member and the sensor may be located on the extension member.

In addition or alternative and in a twenty third aspect, the system may further comprise an elongate member configured to extend to a location distal of a distal end of the catheter, wherein the sensor may be located at a distal end portion of the elongate member.

In addition or alternative and in a twenty fourth aspect, a method of removing contrast from a patient's vascular system may include inserting a sensor into a vessel of a patient at a location spaced from and distal of a distal end of an aspiration lumen in a catheter, wherein the catheter may comprise an expandable member and the aspiration lumen, where the distal end of the aspiration lumen may be distal of the expandable member. The method may further comprise comparing a value sensed by the sensor at the location spaced from and distal of the distal end of the aspiration lumen to a threshold value, initiating expansion of the expandable member in response to a determination that the value reaches and/or is beyond the threshold value, and initiating suction of fluid through the aspiration lumen in response to the determination that the value reaches and/or is beyond the threshold value.

In addition or alternative and in a twenty fifth aspect, the method may further comprise after initiating suction of fluid, stopping the suction of fluid at a predetermined time after determining that the value reaches and/or is beyond the threshold value again.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic diagram of an example contrast removal system positioned within a heart;

FIG. 2 is a schematic diagram of an example contrast removal system positioned within an iliac vein;

FIG. 3 is a schematic diagram of an example contrast removal system with a distal portion of the example contrast removal system shown in cross-section;

FIG. 4 is a schematic diagram of an example contrast removal system with a distal portion of the example contrast removal system shown in cross-section;

FIG. 5 is a schematic diagram of an example contrast removal system with a distal portion of the example contrast removal system shown in cross-section;

FIG. 6 is a schematic diagram of an example contrast removal system with a distal portion of the example contrast removal system shown in cross-section;

FIG. 7 is a schematic diagram of an example contrast removal system with a distal portion of the example contrast removal system shown in cross-section;

FIG. 8A is a schematic diagram of an example contrast removal system in a bypass configuration with a distal portion of the example contrast removal system shown in cross-section;

FIG. 8B is a schematic diagram of an example contrast removal system in an aspiration configuration with a distal portion of the example contrast removal system shown in cross-section;

FIG. 9 is a schematic diagram of a filtering portion of an example contrast removal system; and

FIGS. 10A-17 are a series of schematic diagrams that show example delivery and use techniques of an example contrast removal system to and within a heart.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Medical procedures may rely on or require imaging (e.g., via computed tomography (CT) scans, magnetic resonance imaging (MRI), angiography, fluoroscopy, and/or other imaging techniques) to provide detailed information to medical personnel. In some cases, imaging may include use of contrast media (e.g., radio-dense contrast media or other contrast media) that is injected into biological structures to be imaged. In some cases, the contrast media may be injected into a patient's vasculature via the patient's arterial system prior to the medical imaging procedure, pass to a target location and into the patient's venous system, and then pass into the patient's renal system, which clears the contrast media from the patient's bloodstream.

Contrast induced acute kidney injury (AKI) (e.g., contrast induced nephropathy (CIN) or other kidney related injury) occurs in a about 7% of all patients in which contrast is used for coronary angiography and about 25% or more of those patients that are considered high risk for contrast induced AKI. Patients with peripheral arterial disease frequently have severe comorbidities and may have a similar or higher risk of developing contrast induced AKI (e.g., CIN) when contrast is used for imaging such a patient's periphery. For example, contrast induced AKI occurs in about 10% of all patients in which contrast is used for periphery imaging. Patients considered to be a high risk for contrast induced AKI or comorbidities may include patients with pre-existing kidney related medical issues, patients that have previously received contrast, patients that have diabetes mellitus, patients that have congestive heart failure, patients that are obese, and/or patients having one or more other kidney related medical issues. In some cases, contrast induced AKI may result in a need for dialysis and could result in death if severe and/or not addressed. Due to the seriousness of contrast induced AKI, systems have been developed to reduce and/or mitigate an amount of contrast that travels to a patients kidneys via the bloodstream.

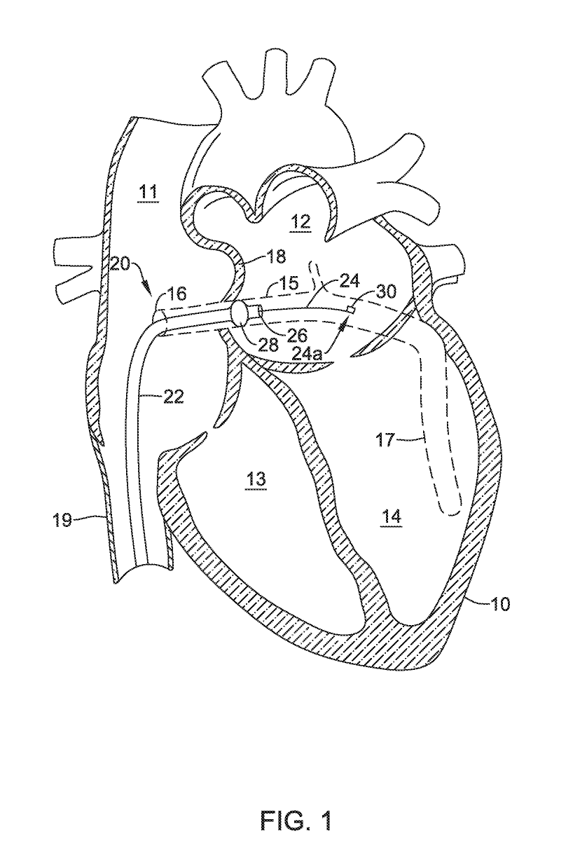

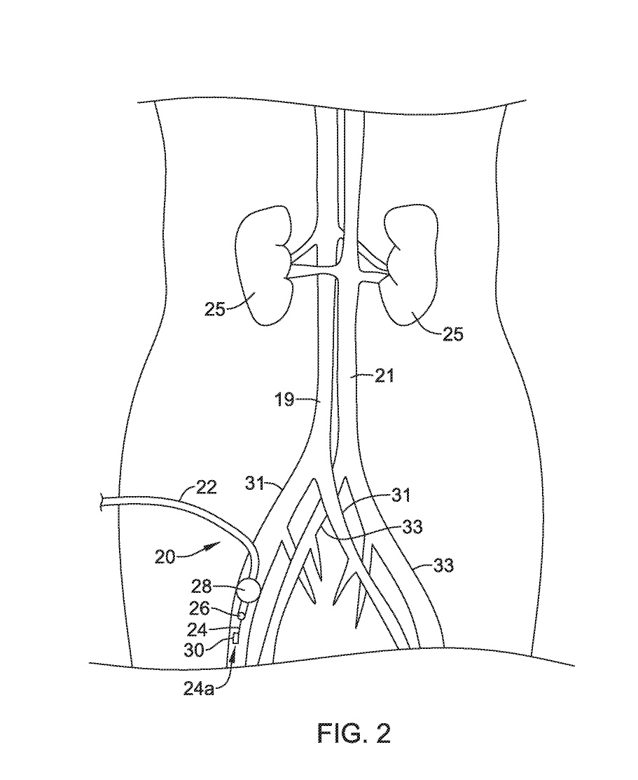

Turning to the Figures, FIG. 1 and FIG. 2 are conceptual diagrams of an illustrative system 20 for removing contrast media from a patient's vasculature. FIG. 1 shows an illustrative catheter 22 and an elongate member 24 inserted into a patient's heart 10. The heart 10 of FIG. 1 is depicted showing a right atrium 11, a left atrium 12, a right ventricle 13, a left ventricle 14, a coronary sinus 15, a coronary sinus ostium 16, a great cardiac vein 17, a septum 18, and an inferior vena cava 19. FIG. 2 shows the illustrative catheter 22 and the elongate member 24 inserted into a patient's iliac vein 31 (e.g., an external iliac vein, an internal iliac vein, a common iliac vein). As shown in FIG. 2, the iliac vein 31 may extend from the patient's inferior vena cava 19 which is connected to the kidneys 25. The iliac vein 31 may be the sole returning path for blood delivered to the patient's leg via an iliac artery 33 extending from an aorta 21. Although FIG. 1 and FIG. 2 depict specific examples of locations within the patient at which the contrast removal system 20 may be used, the contrast removal system 20 may be utilized at other areas of the patient's vasculature, particularly at areas of the patient's vasculature that may be considered a collection area of oxygen-depleted blood or a collection area of blood located downstream of where a contrast is introduced to the patient and upstream of the kidneys 25.

In the example of FIG. 1, the catheter 22 may include a distal end 26 and an expandable member 28. The expandable member 28 may be located proximally of the distal end 26 of the catheter 22. Alternatively, or in addition, the expandable member 28 may extend from the distal end 26 of the catheter 22 and/or form at least a portion of the distal end 26 of the catheter 22. Other configurations of the expandable member 28 relative to the distal end 26 of the catheter 22 are contemplated. In some cases, the catheter 22 may be a balloon catheter having any size diameter. In one example, the catheter 22 may be about a 5 Fr to 8 Fr catheter, however, other sizes of the catheter 22 that facilitate traversing a patient's vascular system (e.g., into the heart, into the venous system around the heart, into the peripheral venous system, and/or one or more other locations in the patient's vascular system) are contemplated.

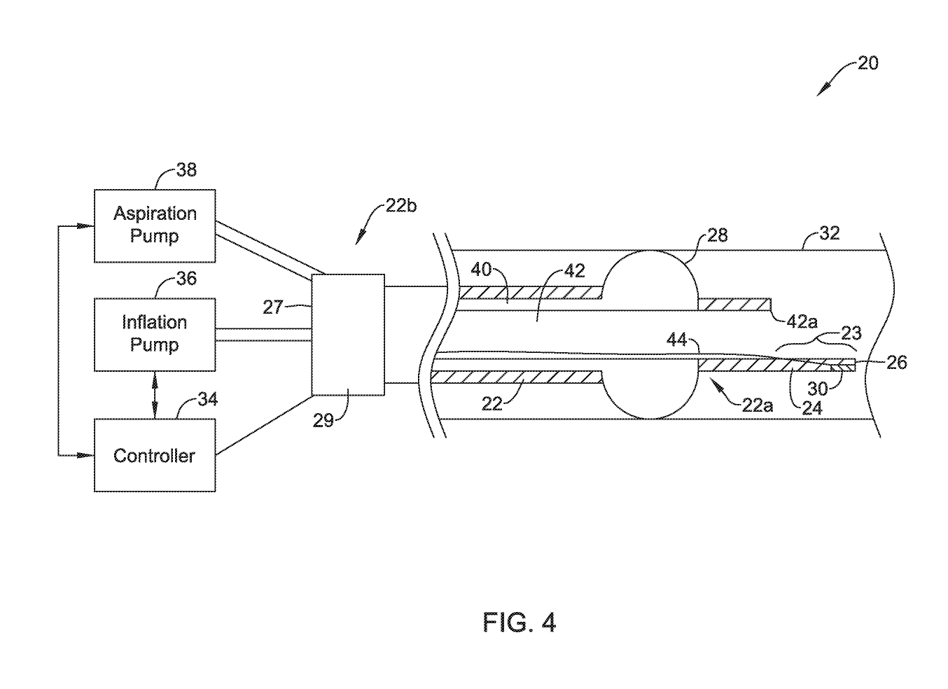

The elongate member 24 may be any type of elongate member. For example, the elongate member 24 may be a wire (e.g., a guide wire or other wire), a catheter, one or more optical fibers, and/or one or more other elongate members. The elongate member 24 may be configured to extend through and/or along the catheter 22 and may have a distal end portion 24a that is configured to extend distally of the distal end 26 of the catheter 22. Alternatively, the elongate member 24 may be an extension of the catheter 22 extending distally of a distal end of a lumen of the catheter 22 (e.g., distally of a distal end 42a of a second lumen 42, as shown in FIG. 4).

In some cases, the elongate member 24 may support a sensor 30. The sensor 30 may be located at any location of the elongate member 24, for example, the sensor 30 may be supported by a distal end portion 24a of the elongate member 24 or other portion of the elongate member 24 such that the sensor may be located distal of a distal end 26 of the catheter 22 or at least distal of distal ends of one or more lumens of the catheter 22. In one example, the sensor 30 may be located on or in the distal end portion 24a of the elongate member 24 at a location that extends distally of a distal end of a lumen of the catheter 22. Further, although the sensor 30 may be primarily described as a single sensor herein, the sensor 30 may comprise a plurality of sensors or sub-sensor, unless clearly indicated otherwise.

In operation, the sensor 30 may be configured to sense a value of a parameter in a bloodstream of a patient at a location distal of the distal end 26 of the catheter 22 or distal of at least a portion of the catheter 22. In some cases, the sensor 30 may be configured to continuously sense a parameter in a bloodstream of a patient. Alternatively, the sensor 30 may be configured to sense a parameter in a bloodstream of a patient at one or more intervals. The parameter may be a percent concentration of contrast in the bloodstream or other parameter. Further, although the sensor 30 may be configured to sense the parameter directly, the sensor 30 may be alternatively or additionally configured to sense a value of a measure related to the parameter (e.g., a measure from which the parameter may be determined).

If a value of the parameter or a value of the measure related to the parameter in the bloodstream of a patient reaches and/or is beyond a threshold value (e.g., an aspiration threshold value), an aspiration pump may be triggered to either start or stop aspirating or suctioning fluid in the bloodstream through a lumen of the catheter 22. Additionally or alternatively, if a value of the parameter or a value of the measure related to the parameter in the bloodstream of a patient reaches and/or is beyond a threshold value (e.g., an expansion threshold value), the expandable member 28 may be configured to adjust between a collapsed configuration and an expanded configuration. In some cases, the aspiration threshold value may be the same as the expansion threshold value. Alternatively, the aspiration threshold value may be different than the expansion threshold value.

As used herein, the terms “goes beyond” and variations thereof are intended to mean any instance in which a sensed value changes from a previously sensed value on one side of a threshold value to a value on an opposite side of the threshold value, regardless of whether the sensed value is above or below the threshold. Thus, a value that is rising over time may reach and/or go beyond a threshold (e.g., an upper or lower threshold) and a value that is falling over time may reach and/or go beyond a threshold (e.g., an upper or lower threshold), where the thresholds may be the same threshold or may be different thresholds.

The threshold values of the parameter and/or measure related to the parameter may be any value determined to be a threshold. Further the threshold values may be predetermined, determined based on a patient baseline value from the sensor 30, determined based on a distance the sensor is from a feature of the catheter 22, and/or determined based on one or more other factors. The threshold values may be in terms of the parameter and/or in terms of values of the measure related to the parameter.

In some cases, a threshold value for initiating aspiration or suction of fluid in the bloodstream and/or expanding the expandable member 28 may be a value indicative of a contrast concentration level in the bloodstream of greater than about 1%, greater than about 5%, greater than about 10%, greater than about 20%, greater than about 30%, greater than about 40% or less other concentration level. Further, a threshold value for stopping aspiration or suction of fluid in the bloodstream and/or collapsing the expandable member 28 may be a value indicative of a contrast concentration level in the bloodstream of less than about 40%, less than about 30%, less than about 20%, less than about 10%, less than about 5%, less than about 1%, or less than any other concentration level. In one example, a threshold value for initiating aspiration or suction of fluid in the bloodstream and/or expanding the expandable member 28 may be a value indicative of a contrast concentration level in the bloodstream of greater than a level between about 30% and 40%, and a threshold value of initiating stopping aspiration or suction of fluid in the bloodstream and/or collapsing the expandable member 28 may be a value indicative of a contrast concentration level in the bloodstream of less than about 5%.

The sensor 30 may be any type of sensor configured to sense values of a parameter and/or a measure related to a parameter of a patient's bloodstream. In some cases, as discussed above, sensed values from the sensor 30 may be, or may be indicative of, a contrast concentration in a bloodstream or other parameter of the bloodstream.

Measures related to parameters of the bloodstream may have values that are indicative of a value of a parameter of the bloodstream, such as a value that is indicative of a contrast concentration in the bloodstream or other parameter. Examples of measures related to parameters in the bloodstream may include, but are not limited to, impedance of the blood stream, thermal dilution of the bloodstream, wavelengths of the of the blood stream, viscosity of the bloodstream, chemical make-up of the blood stream, pH level of the bloodstream, density of the blood, and/or one or more other parameters.

As discussed, values of measures related to the parameter that are sensed by the sensor 30 may be indicative of a concentration level or an amount of contrast in a bloodstream of patient. For example, values of measures related to the parameter may have a positive correlation with a concentration level or an amount of contrast in fluid of the bloodstream (e.g., increasing values and decreasing values of the measures related to the parameter are indicative of increasing and decreasing, respectively, concentration levels or amounts of contrast in the fluid of the blood stream) or a negative correlation with a concentration level or an amount of contrast in blood of the bloodstream (e.g., decreasing and increasing values of the measures related to the parameter are indicative of increasing and decreasing, respectively, concentration levels or amounts of contrast in the blood of the bloodstream).

In one example of the sensor 30, the sensor 30 may be configured to sense a conductivity (e.g., impedance) of fluid in the bloodstream. A sensor 30 configured to sense conductivity of fluid in the bloodstream may include one or more pairs of electrodes. In some cases, the sensor 30 may include multiple pairs and/or an array (e.g., multiple rows and/or columns) of electrodes to facilitate sensing a measure related to conductivity (e.g., impedance or other measure) of fluid in the bloodstream over time, which may correlate with a concentration of contrast level in the fluid of the bloodstream. Multiple pairs and/or an array of electrodes may reduce potential false readings (e.g., which may occur if the sensor were to come into contact with patient tissue).

Another example sensor 30 may be configured to sense thermal dilution of fluid in the bloodstream. A sensor 30 configured to sense thermal dilution may include a thermistor or other temperature sensor. The sensor 30 may use the thermistor or other temperature sensor to sense temperature over time and as a volume and temperature of contrast injected into a patient may be known, the temperature sensed by the sensor 30 may be correlated with an amount of contrast in the fluid of the bloodstream.

Another example sensor 30 may be configured to sense a color change of fluid in the bloodstream. A sensor 30 configured to sense a color change may include a fiber optic sensor or other optical sensor. When the sensor 30 includes a fiber optic sensor, the elongate member 24 may be one or more optical fibers and/or may include one or more optical fibers at the distal end portion 24a of the elongate member 24. The sensor 30 may use the optical fibers or other optical sensor elements to track a wavelength of light reflected from the fluid in the bloodstream over time, which may be correlated with an amount of contrast in the fluid of the bloodstream.

As discussed above, other configurations of the sensor 30 may sense parameters and/or measures related to one or more additional or other parameters of fluid in the bloodstream. In some cases, one or more sensors 30 may be provided to sense two or more measures related to a parameter of fluid in a bloodstream to increase sensitivity and/or specificity of determining a contrast concentration level in a bloodstream.

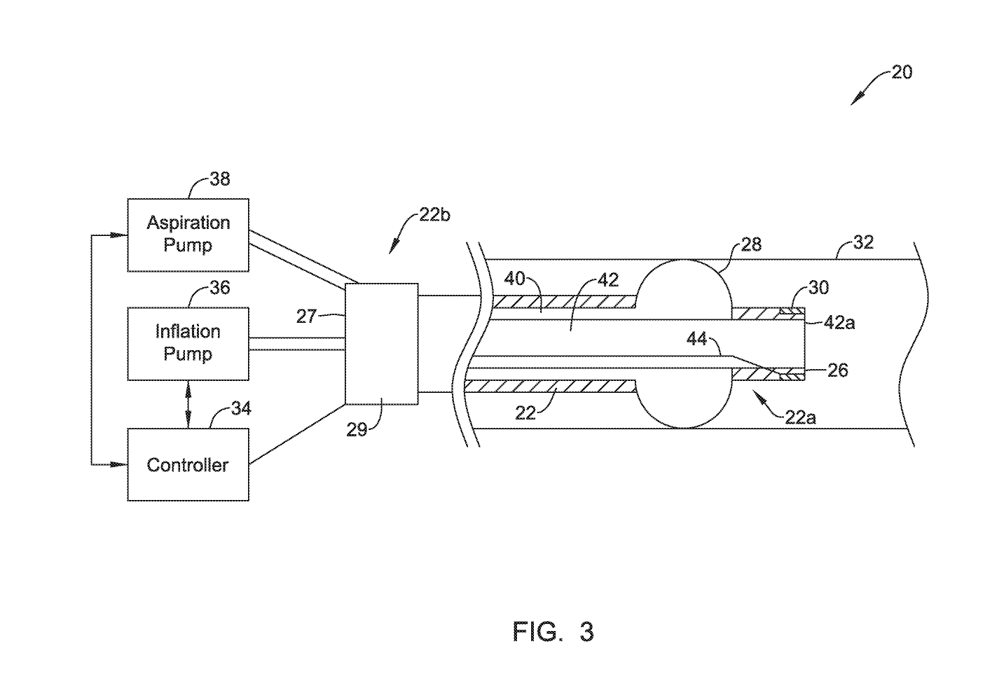

FIGS. 3-8B depict various configurations of contrast removal systems 20 at least partially inserted into a vessel 32 (e.g., the coronary sinus 15, iliac vein 31, etc.) of a vascular system of the patient, where the portion of the contrast removal system 20 located within the vessel 32 is shown in cross-section. Further, as discussed in greater detail below, although the expandable member 28 is depicted in FIGS. 3-8B in an expanded configuration, the default configuration of the expandable member 28 may be a collapsed configuration to facilitate delivery of the catheter 22 to a target location (e.g., within the coronary ostium, iliac vein, or other location in the patient's vascular system) and/or allow fluid of the bloodstream to pass by catheter 22 when a prerequisite amount of contrast is not present in the fluid.

As depicted in FIGS. 3-8B, the contrast removal system 20 may include the catheter 22 having an expandable member 28 and the sensor 30. In some cases, the contrast removal system 20 may further include a controller 34, a first pump 36 (e.g., an inflation pump or other pump), a second pump 38 (e.g., an aspiration or suction pump or other pump), and/or one or more other features. Note, elements of the contrast removal system 20 present in each of FIGS. 3-8B are described generally with respect to FIGS. 3-8B, but may not be particularly discussed with respect to each individual figure.

The catheter 22 may have the distal end 26 at a terminal end of a distal end portion 22a of the catheter 22, a proximal end 27 at a terminal end of a proximal end portion 22b of the catheter 22, and the expandable member 28 located between the distal end 26 and the proximal end 27. In some cases, the catheter may have a hub 29 that at least partially defines the proximal end 27. The hub 29, when included, may provide ports for connecting to one or more of the controller 34, the first pump 36, the second pump 38, and/or one or more other features of the system 20. Alternatively or in addition, the hub 29 may include one or more of the controller 34, the first pump 36, the second pump 38, one or more other features, and an actuation mechanism or control(s) for interacting with one or more of the features connected to and/or included with the hub 29.

As shown in FIGS. 3-8B, the expandable member 28 of the catheter 22 may be proximal of and adjacent to the distal end 26 of the catheter 22. However, the expandable member 28 may extend from and/or form a portion of the distal end 26 of the catheter 22. Alternatively or in addition, the expandable member 28 may be located at one or more other locations of the catheter 22 such that the expandable member 28 is configured to occlude the vessel 32 when in an expanded configurations.

The expandable member 28 may be any type of expandable member capable of adjusting between a collapsed or delivery configuration and an expanded configuration. In some cases, the expandable member 28 may be a balloon, two or more balloons, two or more balloon portions, an electrically stimulated expandable member, a self-expanding expandable member (e.g., the expandable member 28 may automatically expand when a cover or sheath is removed), and/or other expandable member configured to occlude a vessel in which it is located.

In instances when the expandable member 28 is a balloon or other inflatable structure, the catheter 22 may include a first lumen 40 (e.g., an inflation lumen or other lumen) extending between the first pump 36 and the expandable member 28. Although the first lumen 40 is shown as being co-axial with a portion of the second lumen 42, this is not required. In some cases, the first pump 36 may be in fluid communication with the first lumen 40 and the expandable member 28, such that fluid from the first pump 36 may be provided through the first lumen 40 to adjust the expandable member 28 from the collapsed configuration to the expanded configuration. Additionally or alternatively, the first pump 36 with draw fluid from the expandable member 28 through the first lumen 40 to adjust the expandable member 28 from the expanded configuration to the collapsed configuration. The first pump 36 may be any type of pump configured to pump fluid to and from the expandable member 28.

The catheter 22 may include the second lumen 42 (e.g., an aspiration lumen, a suction lumen, a bypass lumen, or other lumen). The second lumen 42 may extend an entire length between the distal end 26 of the catheter 22 and the proximal end 27 of the catheter 22. Alternatively or in addition, the second lumen 42 may extend between a distal end 42a of the second lumen 42 to the proximal end 27 of the catheter and/or a proximal end (not shown) of the second lumen 42. In some cases, the distal end 42a and the proximal end of the second lumen 42 may be at the distal end 26 and the proximal end 27, respectively, of the catheter 22. Alternatively, the distal end 42a of the second lumen 42 may be proximal of a distal end 26 of the catheter 22 and/or the proximal end of the second lumen 42 may be distal of the proximal end 27 of the catheter 22.

The controller 34 may be any type of controller. In some cases, the controller may include one or more processors and memory, which may be configured to coordinate operation of various electronic features (e.g., the sensor 30, the first pump 36, the second pump 38, and/or other electronic features) of the system 20. Further, the controller 34 may include a user interface having one or more of buttons, screens (e.g., touch screens or non-touch screens), microphones, speakers, lights, and other features configured to output information to users and/or take input from users. Alternatively, the controller 34 may not include a user interface or may have a limited user interface and may be configured to communicate via conducted signals, radio frequency (RF) signals, optical signals, acoustic signals, inductive coupling, and/or any other suitable communication methodology on a wired or wireless (e.g., through wireless communication protocols including, but not limited to, WiFi, Bluetooth™, Bluetooth Low Energy, Zigbee, etc.) connection with one or more other control devices having a user interface.

The controller 34 may be in electrical communication with one or more other features of the system 20. For example, the controller 34 may be in electrical communication via conducted signals, radio frequency (RF) signals, optical signals, acoustic signals, inductive coupling, and/or any other suitable communication methodology on a wired or wireless (e.g., through wireless communication protocols including, but not limited to, WiFi, Bluetooth™, Bluetooth Low Energy, Zigbee, etc.) connection with the sensor 30, the first pump 36, the second pump 38, and/or one or more other features of the system 20.

In some cases, the controller 34 may be incorporated into one or more of the hub 29, when included, the catheter 22, the sensor 30, the first pump 36, the second pump 38, and/or other feature of the system 20. In one example, the controller 34, the first pump 36, and the second pump 38 may be incorporated into a single device or the controller 34 may be incorporated into one of the first pump 36 and the second pump 38. Alternatively or in addition, as discussed above, the controller 34 may be incorporated into the hub 29 of the catheter 22.

Generally and as discussed in greater detail below, the sensor 30 may sense one or more values of a parameter and/or one or more values of a measure related to the parameter, the sensed values may be communicated to the controller 34, and/or the controller 34 may automatically control the operation of the first pump 36 and/or the second 38 based, at least in part, on the sensed values. Further, in some cases, the operation of one or more of the first pump 36 and the second pump 38 may be manually controlled in response to an alert or other indication from the controller 34 that may be based, at least in part, on the values of the measure related to the parameter(s) sensed by the sensor 30.

In operation, the controller 34 may be configured to receive values of the parameter or the measure related to the parameter sensed by the sensor 30. Further, the controller 34 may include and/or may establish (e.g., based on baseline readings from a sensor or other input to the controller 34) one or more threshold values for the parameter or the measure related to the parameter and store the threshold values in memory. Then, when one or more values for the parameter or the measure related to the parameter reach and/or are beyond the threshold values, the controller 34 may take one or more actions. In addition or alternatively, when a value of the parameter or the measure related to the parameter reaches and/or is beyond a threshold value, the controller may initiate an alarm and in response, a user (e.g., a physician or other user) may know to turn on or off (or take a different action) one or both of the first pump 36 and the second pump 38. In such cases and/or in other cases, when a value of the parameter or the measure related to the parameter reaches and/or is beyond a threshold value, the controller 34 may automatically turn on and/or off one or both of the first pump 36 and the second pump 38. In some cases, the controller 34 may have at least two threshold values stored in memory and when a value of the parameter or the measure related to the parameter reaches and/or is beyond a first threshold value, the controller 34 may turn on or off the second pump 38 and when a value of the parameter or the measure related to the parameter reaches and/or is beyond a second threshold value, the controller 34 turn on or off the first pump 36.

Turning to FIG. 3, the sensor 30 of the system 20 may be configured to be disposed distally of the expandable member 28 of the catheter 22. As shown in FIG. 3, the sensor 30 may be located on the distal end portion 22a of the catheter at a location adjacent to and distal of the expandable member 28. In the example of FIG. 3, the sensor 30 may be supported by the catheter 22 at or adjacent the distal end 26 of the catheter 22 and the distal end 42a of the second lumen 42. When so positioned, a wire 44 or lead may extend proximally from the sensor 30 to the hub 29 of the catheter 22 and/or the controller 34 to provide values of a parameter or a measure related to a parameter. Although not shown, the sensor 30 may communicate with the controller 34 in a wireless manner.

FIG. 4 depicts a configuration of the system 20 similar to the system depicted in FIG. 3, however, the catheter 22 includes an extension portion 23 with the sensor 30 located on the extension portion 23. In some cases, the extension portion 23 of the catheter 22 may extend from the distal end 42a of the second lumen 42 to the distal end 26 of the catheter 22, as shown in FIG. 4, and the sensor 30 may be located at or adjacent the distal end 26 of the catheter 22. Although not necessarily shown in FIG. 4, the extension portion 23 may be configured to extend into a vein extending from or leading to the coronary sinus 15.

The extension portion 23 of the catheter 22 extending distally of the distal end of the second lumen (e.g., the aspiration lumen) with the sensor 30 located at the distal end 26 of the catheter 22 may allow for the sensor 30 to sense measures related to parameters within the vessel 32 (e.g., the coronary sinus 15, the great cardiac vein 17, or other vessel extending from or in communication with the coronary sinus 15) at a location distally spaced from a distal end of the distal end 42a of the second lumen 42. Such a configuration of the system 20 may allow time for adjusting the expandable member 28 to an expanded configuration and/or turning on the second pump 38 (e.g., the aspiration pump) prior to contrast reaching the distal end 42a of the second lumen 42. As a result, such sensing the parameters upstream of the distal end 42a of the second lumen may allow for reducing an amount of contrast passing to a patient's kidneys 25 when compared to systems having a sensor located proximal of the distal end 42a of the aspiration lumen 42.

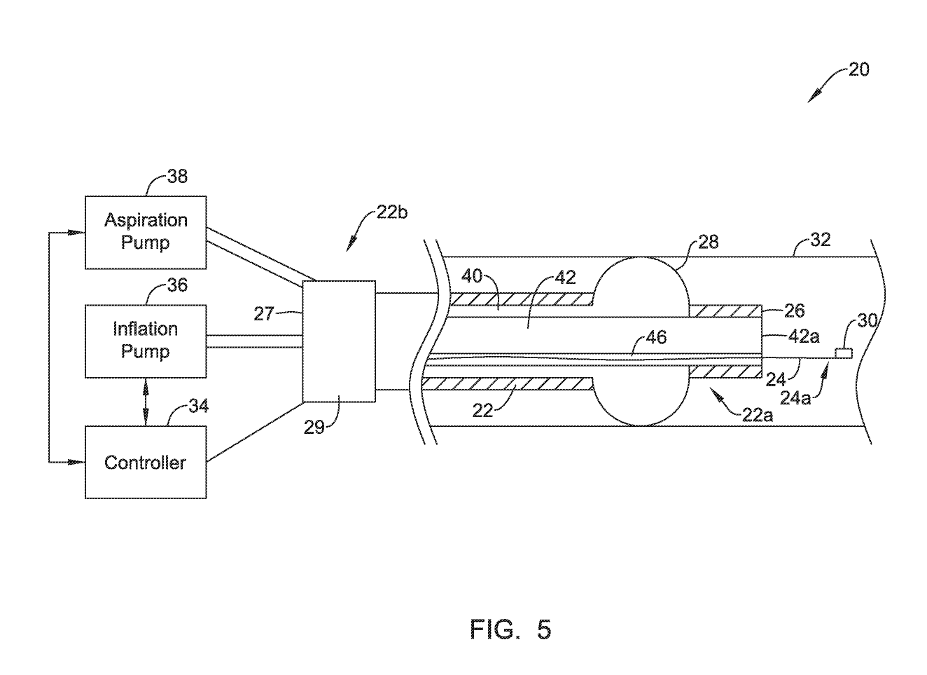

FIGS. 5-8B depict various systems 20 that may allow for positioning the sensor 30 at a location distal and/or upstream of the distal end 42a of the second lumen 42, adjustability of a distance between the sensor 30 and the distal end 42a of the second lumen 42, and maneuverability of the distal end portion 24a of the elongate member 24 to facilitate positioning the elongate member 24 and/or the sensor 30 in the patient's vasculature (e.g., the coronary sinus 15, the iliac vein 31, etc.) As bloodstreams of various patients may have different flow rates and/or different contrast materials may flow at various rates depending on quantity of contrast, type of contrast, and/or other factors, the ability to adjust a distance between the sensor 30 and the distal end 42a of the second lumen 42 and maneuver the sensor 30 into various vessels of a patient's vasculature (e.g., vessels leading into the coronary sinus 15, vessels leading into the iliac vein 31, etc.), allows for physicians and/or the controller 34 to precisely remove contrast from the patient's bloodstream while mitigating an amount of blood removed from the bloodstream that does not include contrast.

The elongate member 24 may be a wire or at least a partially tubular structure. The elongate member 24 may be a guide wire or a guide catheter, but this is not required.

In some cases, the elongate member 24 may be (e.g., when a wire) or may include an electrically conductive feature that facilitates electrical communication between the sensor 30 and the controller 34. When so configured, the elongate member 24 may connect directly into the controller 34, the elongate member 24 may connect to the hub 29 of the catheter and the hub 29 may include a feature that communicates with the controller 34, the elongate member 24 may connect to or include another feature of the system 20 that is in communication with the controller 34, and/or the sensor 30 and the elongate member 24 may communicate with the controller 34 in one or more other manners.

To facilitate maneuverability and/or for other purposes, the elongate member 24 may be flexible. In some cases, the elongate member 24 may be made of a shape memory material. In such cases, the shape memory material of the elongate member 24 may include one or more pre-formed bends that may cause the elongate member 24 to bend in a desired direction once the elongate member 24 is extending distally of the distal end 26 of the catheter 22. Further, the elongate member 24 may include one or more pull wires or other mechanisms to facilitate directing the elongate member through vessels feeding a bloodstream into the coronary sinus 15 and/or other vessel.

FIG. 5 depicts the system 20 in a configuration that may facilitate the elongate member 24 extending through a third lumen 46 and out the distal end 26 of the catheter 22. The sensor 30 may be located on or supported by the distal end portion 24a of the elongate member 24 and the elongate member 24 may be axially adjustable within the third lumen 46 to allow for adjusting a distance between the distal end 26 of the catheter 22 or the distal end 42a of the second lumen 42 and the sensor 30. In the configuration of the system 20 in FIG. 5, the third lumen 46 may extend between the distal end 26 of the catheter 22 and the proximal end 27 or adjacent the proximal end 27 of the catheter 22. Such a configured catheter 22 may be considered an over-the-wire catheter. In the catheter 22 having an over-the-wire configuration, the elongate member 24 may be within the catheter 22 (e.g., within the third lumen 46 or other lumen) for substantially an entire or an entire length of the catheter 22.

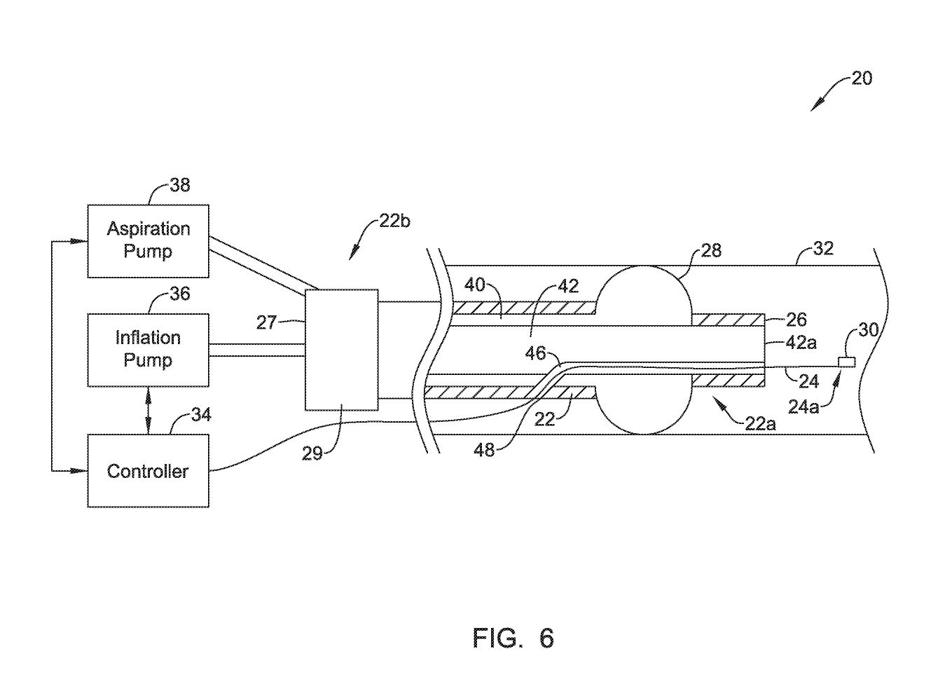

FIG. 6 depicts the system 20 in a configuration that may facilitate the elongate member 24 extending through the third lumen 46 and out of the distal end 26 of the catheter 22. The sensor 30 may be located on or supported by the distal end portion 24a of the elongate member 24 and the elongate member 24 may be axially adjustable within the third lumen 46 to allow for adjusting a distance between the distal end 26 of the catheter 22 or the distal end 42a of the second lumen 42 and the sensor 30. The configuration of the system 20 in FIG. 6 differs from the configuration of FIG. 4 in that the third lumen 46 may extend between the distal end 26 of the catheter 22 and a side port 48 extending through the catheter at a location proximal of the expandable member 28 and distal of the proximal end 27 of the catheter 22. Such a configured catheter 22 may be considered a rapid exchange catheter. In the catheter 22 having the rapid exchange configuration, the elongate member 24 may extend along an exterior surface of the catheter 22, through the side port 48, and through the distal end 26 of the catheter 22. Further, the elongate member 24 may connect directly to the controller 34, as shown in FIG. 6, but this is not required.

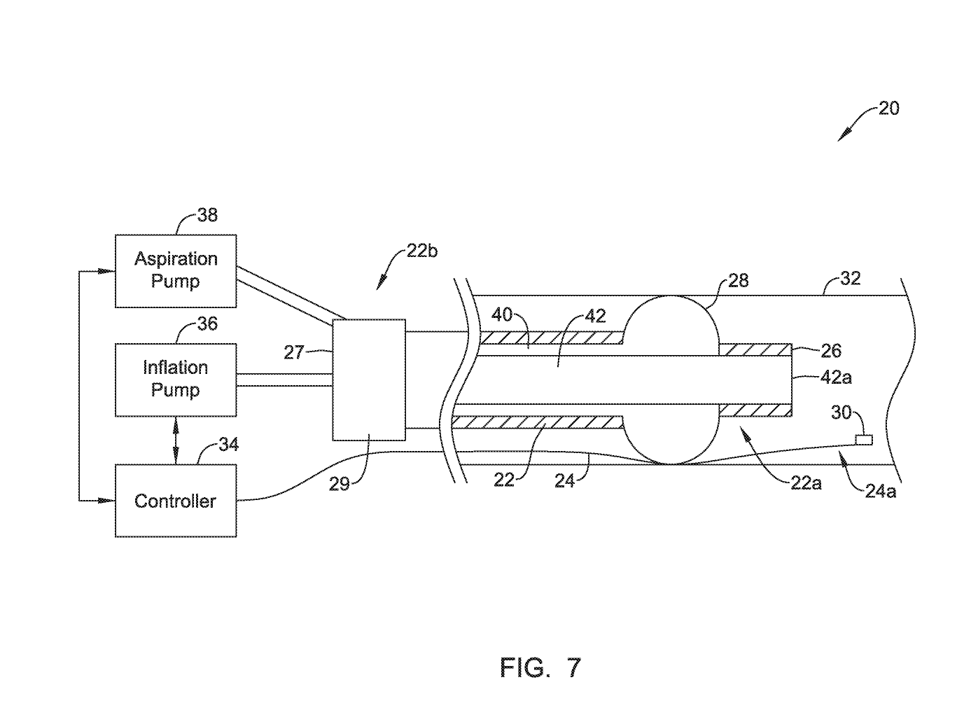

FIG. 7 depicts the system 20 in a configuration that may facilitate the elongate member 24 extending along an outside surface of the catheter 22 to a location distal of the distal end 26 of the catheter 22. The sensor 30 may be located on or supported by the distal end portion 24a of the elongate member 24 and the elongate member 24 may be axially adjustable along the catheter 22 to allow for adjusting a distance between the distal end 26 of the catheter 22 or the distal end 42a of the second lumen 42 and the sensor 30. The configuration of the system 20 in FIG. 7 differs from the configurations of FIGS. 5 and 6 in that the elongate member 24 may not extend through a lumen of the catheter 22 at all and as a result, the catheter 22 may or may not include the third lumen 46. In the system 20 where the elongate member 24 may extend along an exterior of the catheter 22, the elongate member 24 may be positionable substantially or completely independent of a positioning of the catheter 22 or the elongate member 24 and the catheter 22 may be positionable in coordination with one another. Further, the elongate member 24 may connect directly to the controller 34, as shown in FIG. 7, but this is not required.

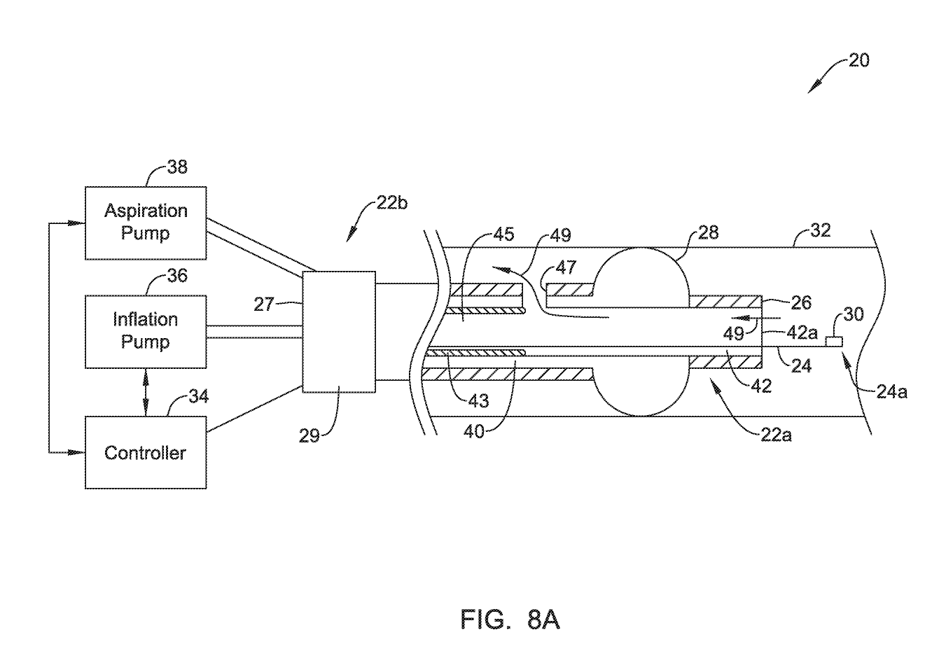

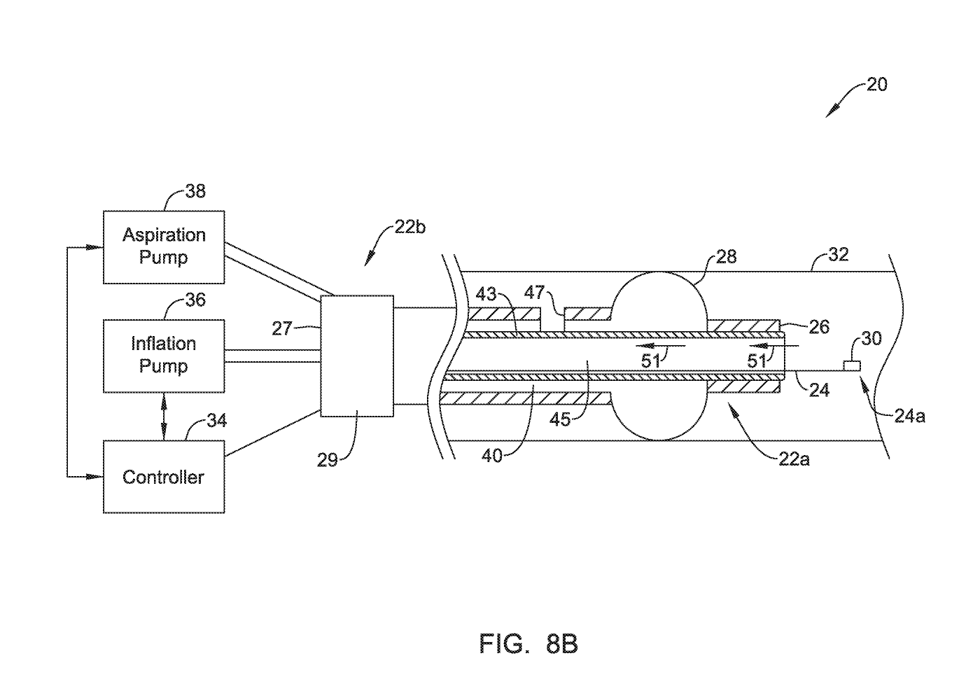

FIGS. 8A and 8B depict a configuration of the system 20 that includes two catheters. As shown in FIGS. 8A and 8B, the system 20 may include the catheter 22 (e.g., an outer catheter) and an inner catheter 43, where the inner catheter 43 and the catheter 22 may be longitudinally and/or rotationally adjustable with respect to one another (e.g., in a tele-sheath relationship).

The inner catheter 43 may be adjusted with respect to the catheter 22 in any manner. For example, the adjusting of the inner catheter 43 relative to the catheter 22 may be done manually and/or in an automated manner. When the inner catheter 43 is adjusted in an automated manner, a motor (e.g., glide motor, stepper motor, or other motor) may be utilized to adjust a longitudinal and/or rotational position of the inner catheter 43. In some cases, an automated arrangement of the catheter 22 and the inner catheter 43 may be a push-pull robotic catheter, but this is not required.

Although the catheter 22 and the inner catheter 43 are referred to herein as separate catheters, these catheters may be considered an outer catheter and an inner catheter respectively of a single catheter. The single catheter may have one or more lumens, as discussed herein, and the one or more lumens may be included in one or both of the catheter 22 and the inner catheter 43.

In some cases, one or both of an outer surface of the inner catheter 43 and an inner surface of the catheter 22 may include a coating that facilitates moving the catheter 22 and the inner catheter 43 with respect to one another (e.g., a hydrophilic coating and/or other coating facilitating relative movement of the catheter 22 and the inner catheter 43). Additionally or alternatively, one or both of an outer surface of the inner catheter 43 and an inner surface of the catheter 22 may include a surface feature that facilitates moving the catheter 22 and the inner catheter 43 with respect to one another (e.g., a groove, a polish, and/or other surface feature facilitating relative movement of the catheter 22 and the inner catheter 43).

A close radial fit of the inner catheter 43 with the catheter 22 and/or a protrusion extending between the inner catheter 43 and the catheter 22 may facilitate preventing or limiting blood from traveling or flowing between inner catheter 43 and the catheter 22. For example, the outer surface of the inner catheter 23 may be configured to be in contact with (e.g., directly or through one or more coatings) an inner surface of the catheter 22 to prevent blood from flowing therebetween. Alternatively or in addition, there may be a gap between the outer surface of the inner catheter 43 and the inner surface of the catheter 22. In such cases and/or other cases, one or both of the outer surface of the inner catheter 43 and the inner surface of the catheter 22 may include a protrusion (not shown) configured to engage the other of the inner catheter 43 and the catheter 22 from which the protrusion does not extend, but this is not required and there may be no protrusions.

The catheter 22 in FIGS. 8A and 8B may be substantially similar to the catheter 22 of other embodiments discussed herein. However, the catheter 22 depicted in FIGS. 8A and 8B may include one or more bypass openings 47 extending through a wall of the catheter 22 and located proximally of the expandable member 28. The bypass openings 47 may be configured to allow blood to bypass an occlusion caused by expansion of the expandable member 28 within the vessel 32.

The inner catheter 43 may be any catheter configured to be longitudinally and/or rotationally adjustable relative to the catheter 22 so as to be capable of covering the one or more bypass openings 47. In some cases, the inner catheter 43 may be an aspiration catheter and may include a lumen 45 in communication with the aspiration 38 for aspirating blood and/or contrast from the vessel 32.

Although FIGS. 8A and 8B depict a particular configuration of the inner catheter 43 and the catheter 22, other configurations are contemplated. In one example, the inner catheter 43 may have an opening that may adjustably align with the bypass opening 47 (e.g., via rotational and/or longitudinal movement) to facilitate allowing blood to bypass the expandable member 28 at times when the openings are aligned and to aspirate blood and/or contrast at other times when the openings are not aligned.

The elongate member 24 having the sensor 30 thereon of the catheter system 20 may extend through the lumen 45. Alternatively, the elongate member 24 may extend through a different lumen of one or both of the catheter 22 and the inner catheter 43 and/or along an exterior of the catheter 22, as shown in FIGS. 5-7. Further, in some cases, the sensor 30 of the catheter system 20 may be located at a distal end of one or both of the catheter 22 and the inner catheter 43, as shown in FIGS. 3 and 4.

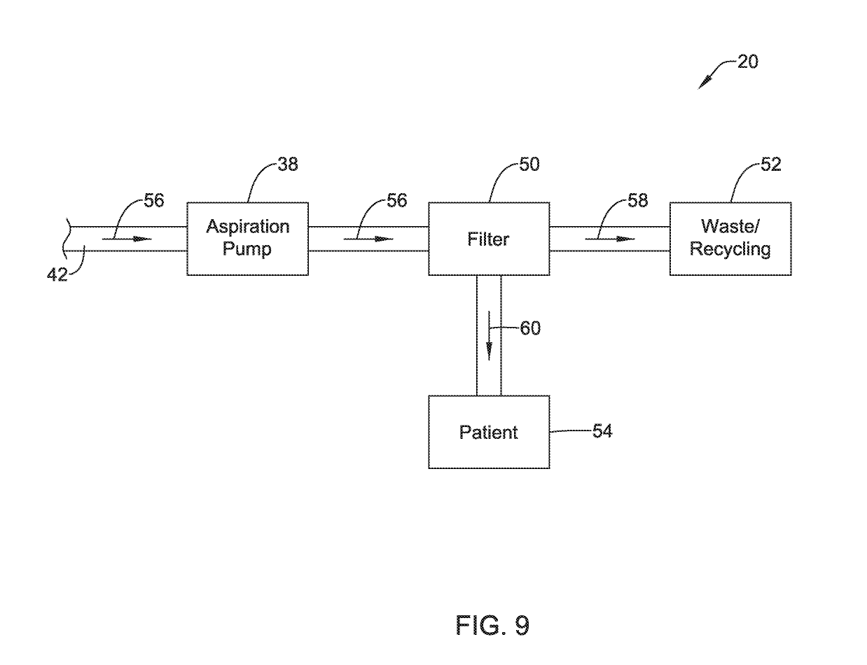

The catheter system 20 of FIGS. 8A and 8B may facilitate anchoring the catheter system 20 at a location adjacent a target location while allowing blood to bypass an occlusion caused by the expanded expandable member 28 anchoring the catheter 22 and reducing the number of steps that need to take place once a certain contrast level has been detected (e.g., removes the step of expanding the expandable member 28). For example, once the distal end 26 of the catheter 22 has been delivered to a target location, the expandable member 28 may be expanded to anchor the catheter 22 at the target location. Although expanding the expandable member 28 may occlude the vessel 32 at the target location, blood in the vessel 32 may flow into the second lumen 42 and out of the bypass opening(s) 47 to bypass the occlusion caused by the expandable member 28, as indicated by arrows 49 in FIG. 8A. When the sensor 30 and/or the controller 34 indicate a contrast level in the blood reaches and/or goes beyond a threshold level, the inner catheter 43 may be moved to a location such that the inner catheter 43 covers the bypass opening 47 (e.g., the inner catheter 43 may be advanced distally to cover the bypass opening 47), as shown in FIG. 8B. As the inner catheter 43 is moved and/or after the inner catheter 43 is moved, the aspiration pump 38 may be initiated to aspirate blood through the lumen 45 of the inner catheter 43, as indicated by arrows 51. Further, once a contrast level in the blood has been determined to be low enough, the aspiration pump may be turned off and the inner catheter 43 may be moved to allow blood to flow through the bypass opening 47 and bypass the occlusion caused by the expanded expandable member 28, while maintaining the expandable member 28 in an expanded configuration. FIG. 9 depicts a portion of the system 20 downstream of the second pump 38 (e.g., an aspiration or suction pump). As depicted in FIG. 9, the second lumen 42 including a mixture 56 of blood and contrast may be passed to the second pump 38. From the second pump 38, the mixture 56 may be passed to a filter 50. The filter 50 may be configured to separate waste fluid 58 from blood fluid 60. From the filter 50, the waste fluid 58 may be provided to a waste or recycling component 52. From the filter 50, the blood fluid 60 may be provided to back to the patient (e.g., patient 54) from which the mixture 56 of blood and contrast was aspirated. Further, although the blood fluid 60 is depicted in FIG. 9 as traveling directly from the filter 50 to the patient 54, this is not always the case and the blood fluid 60 may be further filtered and/or processed. Such a configuration may facilitate aspirating the mixture 56 from the patient while mitigating blood from a blood bank or other source that may be needed to replace the blood aspirated from the patient.

The filter 50 may be any type of filter that is configured to separate blood fluid 60 from another fluid and/or material (e.g., waste fluid 58). In some cases, the filter 50 may be configured such that the blood fluid 60 exiting the filter 50 may be primarily red blood cells and the waste fluid 58 exiting the filter 50 may be may include the contrast material and blood plasma.

Further, although the filter 50 in FIG. 9 is depicted as a single component, the filter 50 may be or may include two or more components. Additionally or alternatively, the filter 50 and the second pump 38 may be combined into a single device.

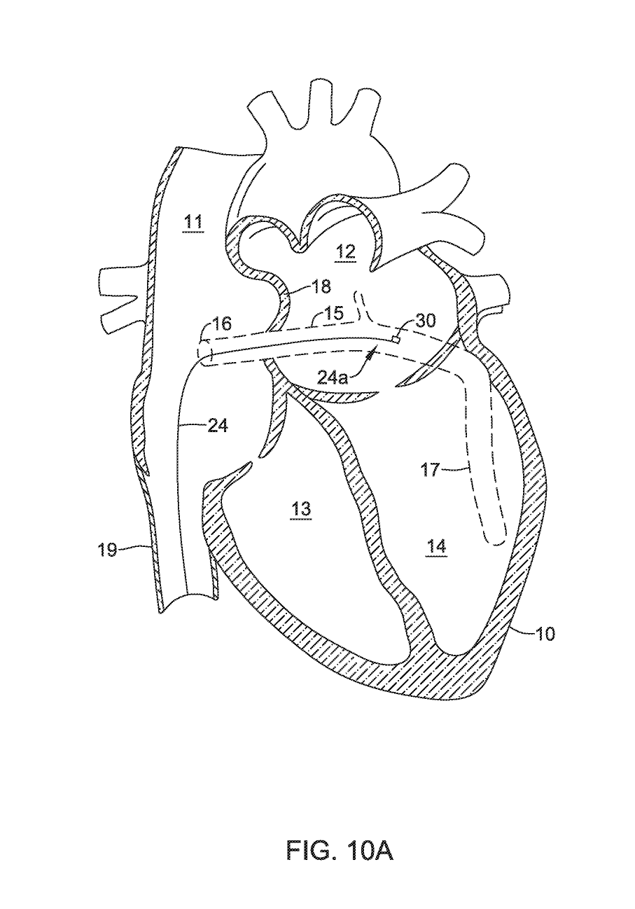

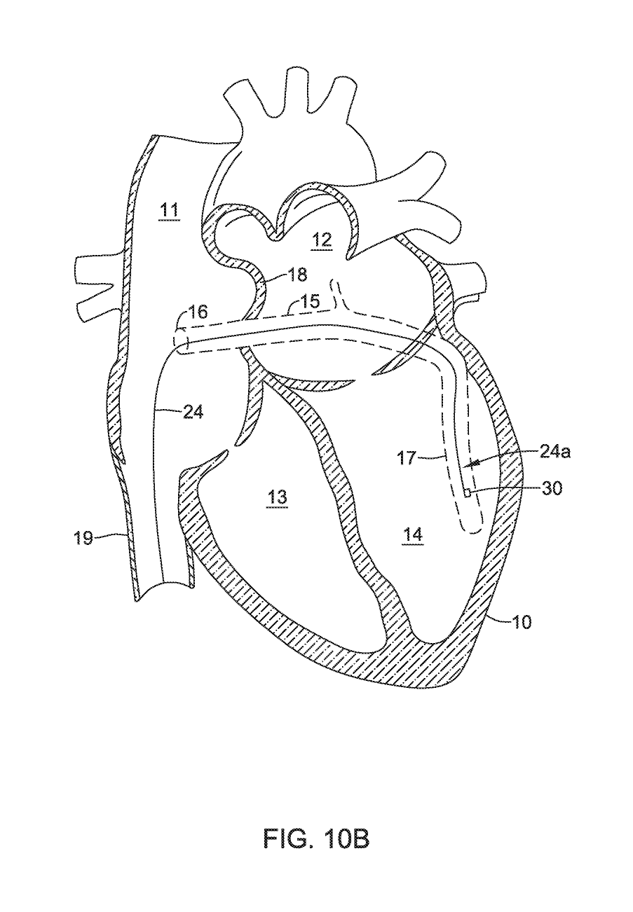

FIGS. 10A-17 depict an example use of the system 20 within the heart 10. Although the example use the system 20 is depicted with respect to the heart 10, similar techniques may be applied at other vessels of a patient's vasculature including, but not limited to, an iliac vein, a jugular vein, etc.

Similar to as shown in FIG. 1, the heart 10 in FIGS. 10A-17 is depicted showing a right atrium 11, a left atrium 12, a right ventricle 13, a left ventricle 14, a coronary sinus 15, a coronary sinus ostium 16, a great cardiac vein 17, a septum 18, and an inferior vena cava 19. Although the depicted use includes obtaining access to the patient's heart 10 through the inferior vena cava 19, access to the heart 10 may also or alternatively be obtained through the superior vena cava and/or other approaches.

In some embodiments, positioning the distal end portion 24a of the catheter and the sensor 30 within the heart 10 may begin by positioning a guide wire or an elongate member within the heart 10, such as the elongate member 24. The elongate member 24 may gain access to the heart 10 through an opening in the patient's skin extending into an artery or vein (e.g., the femoral vein or other vessel) that has been dilated with an introducer or other device having a dilation feature and advancing the elongate member 24 to and/or through the inferior vena cava or other body vessel.

In some instances, the elongate member 24 may have one or more radiopaque markers disposed on an end of the elongate member 24. Such radiopaque markers may allow for easier viewing of the elongate member 24 through one or more medical imaging systems as the elongate member 24 is maneuvered into position within the heart 10. In some embodiments, the radiopaque markers may be spaced apart from each other by a known distance. In such embodiments, by counting the number of radiopaque markers between two features within the heart 10, a distance may be determined between the two features. Based, at least in part, on a determined distance between different features of the patient's heart 10, a distance between the sensor 30 on the elongate member 24 and the distal end 26 and/or the distal end 42a of the second lumen 42 of the catheter 22 may be determined to give the system 20 enough time to adjust the expandable member 28 to an expanded state and initiate aspiration prior to contrast reaching the distal end 42a of the second lumen 42. Additionally or alternatively, the radiopaque marks may facilitate confirming a distance between the sensor 30 and the distal end 26 and/or the distal end 42a of the second lumen of the catheter 22.

After measuring distances between various features of the heart 10, or in embodiments where such measurements are not needed, a distal end portion 24a of the elongate member 24 may be positioned within the coronary sinus 15, as depicted in FIG. 10A. In some instances, the distal end portion 24a of the elongate member 24 and the sensor 30 may be maneuvered all the way through the coronary sinus 15 and into the great cardiac vein 17 (as shown in FIG. 10B) or other vessel extending from the coronary sinus 15. Positioning the distal end portion 24a of the elongate member 24 and the sensor in the great cardiac vein 17 or other vessel extending from the coronary sinus 15, as shown in FIG. 10B, may facilitate creating more distance between the sensor 30 and the distal end 26 of the catheter 22 and/or the distal end 42a of the second lumen 42 of the catheter 22. The positioning of the sensor 30 within the coronary sinus 15 or a vessel extending from the coronary sinus 15 may be determined based at least in part on an amount of time need to actuate the expandable member 28 and the second pump 38, which may be related to a distance between the sensor 30 and distal end 42a of the second lumen 42.

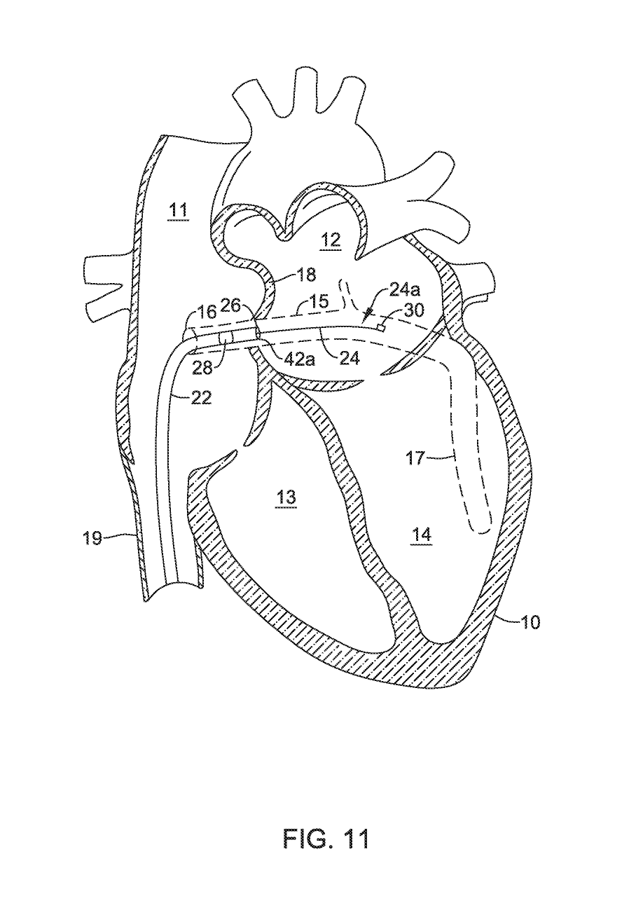

Once the sensor 30 and/or the elongate member 24 are in place, the catheter 22 may be maneuvered over the elongate member 24 into place within the heart 10. Alternatively, a guide wire in addition to or as alternative to the elongate member 24 may be positioned within the coronary sinus 15 and the catheter 22 may be maneuvered over the guide wire and into place within the heart 10. FIG. 11 depicts the catheter 22 advanced to and positioned within the coronary sinus 15. In some cases, the catheter 22 may have a dilator feature (not shown) at or adjacent the distal end (e.g., at or adjacent a distal tip) of the catheter 22. The dilator feature may be configured to engage the ostium 16 of the coronary sinus 15 and dilate and/or cannulate the coronary sinus 15 such that the catheter 22 may be received therein. In one example, the dilator feature of the catheter 22 may take on the structure of a conical tapered tip, such that advancing the catheter 22 into the coronary sinus 15 may expand the inner diameter of the coronary sinus 15. In another example, the dilator feature may be rounded or may have a more abrupt taper than a conical taper. Other dilator feature configurations are contemplated and any configuration suitable for dilating the coronary sinus 15 may be utilized. Alternatively or in addition, a dilating catheter or other dilating device may be utilized to dilate the coronary sinus ostium 16 to facilitate inserting the catheter 22 into the coronary sinus 15.

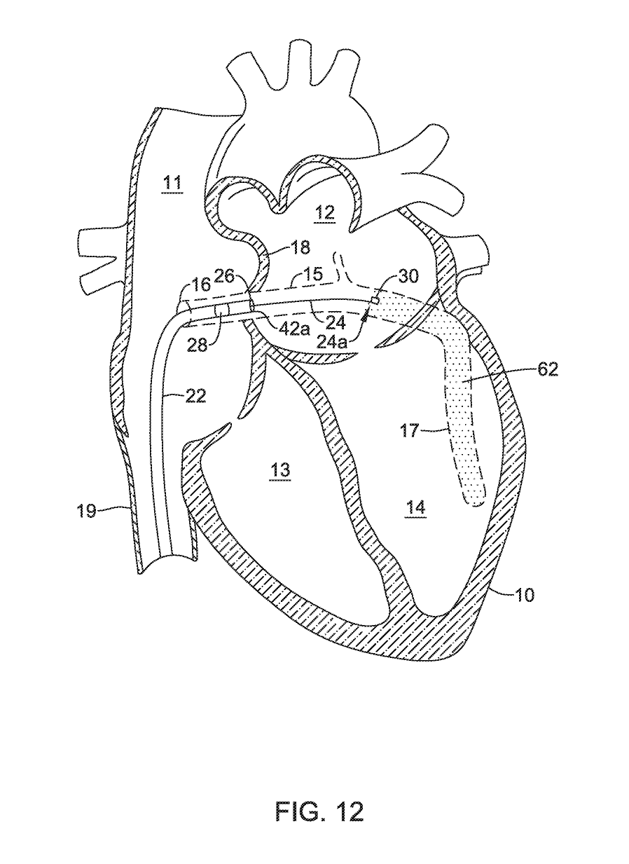

FIG. 12 depicts the catheter 22, the elongate member 24, and the sensor 30 positioned within the coronary sinus 15, where the sensor 30 is placed a distance distal of (e.g., upstream of) the distal end 26 of the catheter 22 and the distal end 42a of the second lumen 42. Although the elongate member 24 is depicted as extending through the catheter 22 in FIGS. 11-17, the elongate member 24 may extend along a side of the catheter 22. In such cases, the elongate member 24 and the catheter 22 may be inserted through the vasculature of a patient independently of one another (e.g., via guide sheath or catheter and/or over one or more wires). Alternatively, the sensor 30 may be positioned on an extension portion 23 of the catheter such that the sensor 30 is distally spaced from a distal end 42a of the second lumen 42.

Once the sensor 30 is positioned within the coronary sinus 15 or a vessel extending from the coronary sinus 15, the controller 34 of the system 20 may cause the sensor 30 to take a baseline reading (e.g., identify a baseline value) for a parameter or a measure related to a parameter at the location of the sensor. Once a baseline reading has been established, one or more threshold values for comparing to values from the sensor 30 of the parameter or the measure related to the parameter may be established and the established threshold values may be saved in memory (e.g., memory of the controller 34 or other memory). Alternatively or in addition, one or more threshold values may be pre-determined and saved in memory (e.g., in memory of the controller 34 or other memory). In some cases, the threshold values may be used to determine when to adjust the expandable member 28 between the collapsed and expanded configurations and/or to determine when to initiate the second pump 38 to aspirate or suction fluid from a vessel.

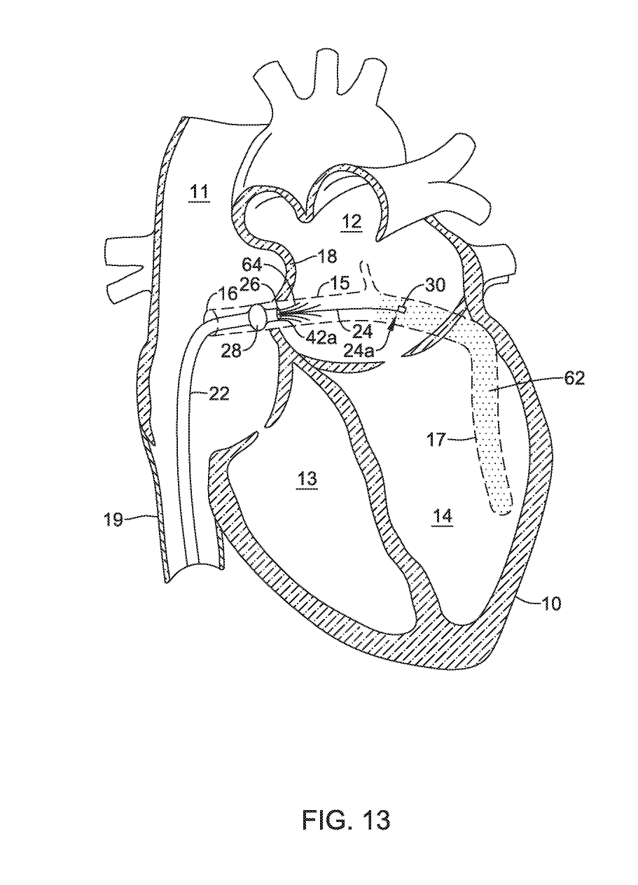

Further, FIG. 12 depicts contrast 62 that has passed through the patient's arterial system, is now in the venous system, and is passing into the coronary sinus 15. Once the contrast 62 reaches the sensor 30, values of the parameter or of the measure related to the parameter that are sensed by the sensor 30 may change and the system 20 may cause the expandable member 28 to adjust from a collapsed configuration (as shown in FIG. 12) to an expanded configuration (as shown in FIG. 13). In one example, once a value of the parameter or the measure related to the parameter reaches and/or is beyond a threshold value, the controller 34 may cause the first and second pumps 36, 38 to actuate and the system 20 may begin aspirating fluid from the coronary sinus 15 and the expandable member 28 may expand to occlude the coronary sinus 15 downstream of the distal end 42a of the second lumen 42. In another example, once a value of the parameter or the measure related to the parameters reaches and/or is beyond a first threshold, the controller may be configured to initiate one of aspiration through the second lumen 42 and adjustment of the expandable member 28 from the collapsed configuration to the expanded configuration. In the example, once the value of the parameter or the measure related to the parameter reaches and/or is beyond a second threshold value, the controller may be configured to initiate the other of aspiration through the second lumen 42 and adjustment of the expandable member 28 from the collapsed configuration to the expanded configuration.

Alternatively, or in addition, to waiting for the value from the sensor 30 of the parameter or the measure related to the parameter to reach and/or go beyond a second threshold, the controller may be configured to wait a time period after initiating one of aspiration through the second lumen 42 and adjustment of the expandable member 28 before initiating the other one of aspiration through the second lumen 42 and adjustment of the expandable member 28, where the time period may be predetermined and/or based on a flow rate of fluid in a bloodstream, a type of contrast used, location of the sensor 30 with respect to the second lumen 42, and/or one or more other factors. In one example, the controller may be configured to wait a time period after initiating adjustment of the expandable member 28 before initiating aspiration through the second lumen 42. This example configuration may facilitate ensuring the coronary sinus 15 is occluded to prevent contrast 62 from passing the catheter 22 before suction begins at the distal end 42a of the second lumen 42. Further, there may be additional or alternative uses of thresholds to modify an operation of the system 20.

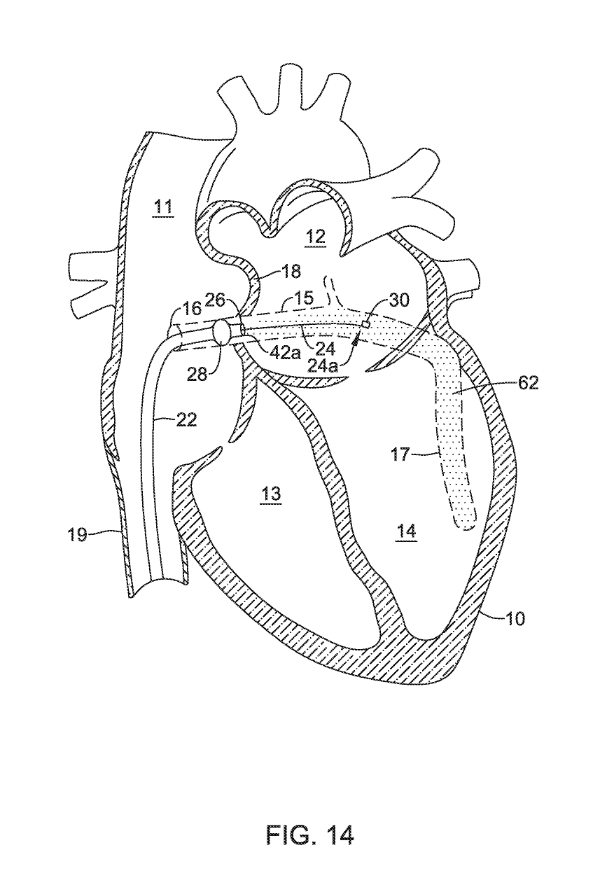

FIG. 13 depicts the contrast 62 flowing towards the distal end 26 of the catheter 22 with the expandable member 28 of the catheter 22 in an expanded configuration and occluding the coronary sinus 15 and the catheter 22 aspirating or suctioning within the coronary sinus 15. The suctioning or aspirating is depicted as suction lines 64 in FIG. 13. FIG. 14 depicts the contrast 62 being suctioned or aspirated into the catheter 22 with little or no contrast 62 passing by the catheter 22. As discussed above with respect to FIG. 9, the contrast 62 may be in a mixture 56 and the mixture 56 may be passed to the filter 50 and separated into waste fluid 58 and blood fluid 60, where the blood fluid may be delivered back to the patient.

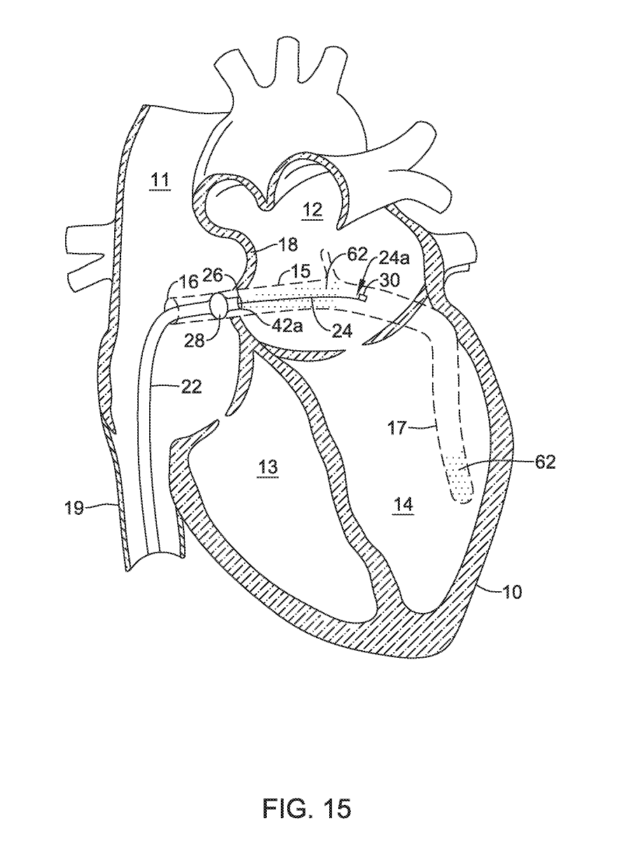

Once the contrast 62 has entirely or substantially passed the sensor 30, the values of the parameter or the measure related to the parameter sensed by the sensor 30 may change and reach and/or go beyond one or more threshold values for determining when to stop suctioning or aspirating and/or for determining when to adjust the expandable member 28 between the expanded configuration and the collapsed configuration. Once such a threshold is reached and/or passed, the controller 34 of the system 20 may be configured to adjust the expandable member 28 from the expanded configuration to the collapsed configuration and stop the second pump from suctioning or aspirating fluid from the coronary sinus 15. In some cases, as shown in FIG. 15, the controller 34 may be configured to delay for a time period after determining values of a parameter or a measure related to a parameter reaches and/or goes beyond a threshold value before adjusting a configuration of the expandable member 28 and/or ceasing or stopping to suction or aspirate through the catheter 22. Such a delay may facilitate removing all or substantially contrast from the bloodstream.

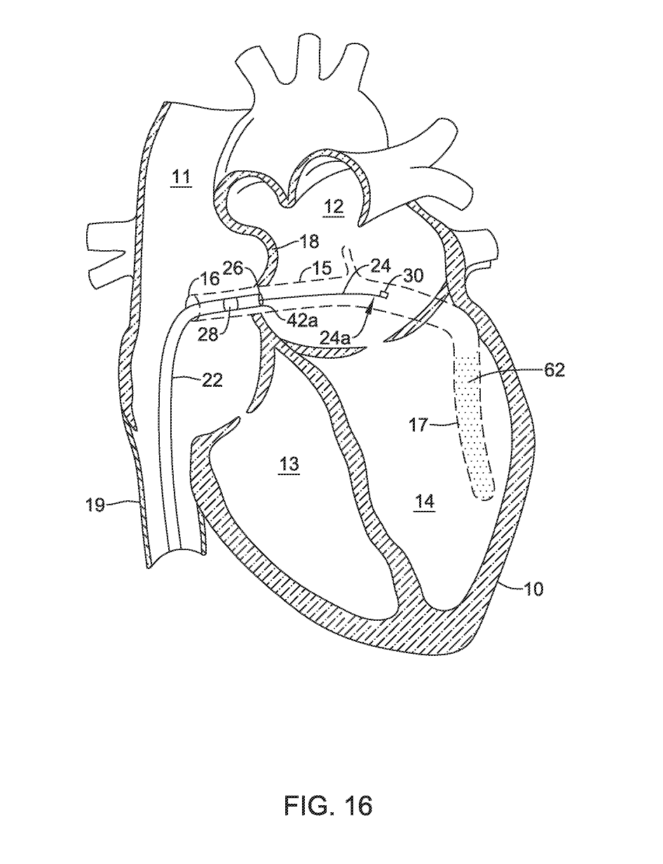

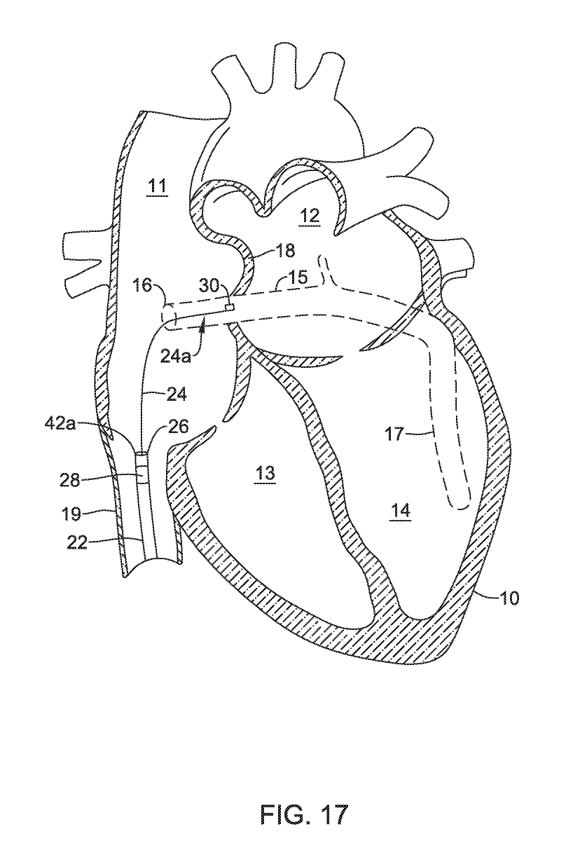

FIG. 16 depicts the catheter 22 after the system 20 determines a value of the parameter or the measure related to the parameter reaches and/or goes beyond a threshold value and the expandable member 28 is adjusted from the expanded configuration to the collapsed configuration and the system 20 is no longer aspirating through the catheter 22. As contrast 62 may be injected into a patient in increments, the distal end of the system 20 may remain in the coronary sinus of a patient, while allowing for the bloodstream to pass by the catheter 22 until a further increment of contrast 62 reaches the sensor 30. Further, once the imaging or other procedure using contrast is over, the catheter 22 and the elongate member 24 may be retracted along the path over which they were inserted into the patient's heart 10, as depicted in FIG. 17.

The materials that can be used for the various components of the system 20 disclosed herein may vary. For simplicity purposes, the following discussion makes reference to catheter 22 and the elongate member 24. However, this is not intended to limit the devices and methods described herein, as the discussion may be applied to other similar members and/or components of the system 20 or components of the delivery systems and procedure systems disclosed herein.

In general, the catheter 22 and the elongate member 24 may be made from any suitable method, and may vary depending on the specific material or materials chosen for the catheter 22 and the elongate member 24. For example, if the catheter 22 and/or the elongate member 24 is made from a metal or metal alloy, the catheter 22 and/or the elongate member 24 may be formed by photo-etching, laser-cutting, micro-machining, 3D printing, sintering, rolled from flat sheet-stock. However, if the catheter 22 and/or the elongate member 24 is made from a polymer material, the catheter 22 and/or the elongate member 24 may be made through extrusion and forming techniques.

The catheter 22 and/or the elongate member 24 and/or other components of system 20, the delivery systems, and/or the procedural systems may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the catheter 22 and/or the elongate member 24 may also be loaded with, made of, or otherwise include a radiopaque material. Radiopaque materials are understood to be materials capable of producing a relatively bright image on a fluoroscopy screen or another imaging technique during a medical procedure. This relatively bright image aids the user of the system 20 in determining its location. Some examples of radiopaque materials can include, but are not limited to, gold, platinum, palladium, tantalum, tungsten alloy, polymer material loaded with a radiopaque filler (e.g., barium sulfate, bismuth subcarbonate, etc.), and the like. Additionally, other radiopaque marker bands and/or coils may also be incorporated into the design of the system 20 to achieve the same result.

In some embodiments, a degree of Magnetic Resonance Imaging (Mill) compatibility is imparted into the system 20. For example, the catheter 22 and/or the elongate member 24 or portions thereof, may be made of a material that does not substantially distort the image and create substantial artifacts (i.e., gaps in the image). Certain ferromagnetic materials, for example, may not be suitable because they may create artifacts in an MRI image. The catheter 22 and/or the elongate member 24, or portions thereof, may also be made from a material that the Mill machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In at least some embodiments, a sheath or covering (not shown) may be disposed over portions or all of the catheter 22 and/or the elongate member 24 that may define a generally smooth outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the catheter 22 and/or the elongate member 24 may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of the catheter 22 and/or the elongate member 24, or other portions of the system 20. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

Those skilled in the art will recognize that the present disclosure may be manifested in a variety of forms other than the specific embodiments described and contemplated herein. For instance, as described herein, various embodiments include one or more modules described as performing various functions. However, other embodiments may include additional modules that split the described functions up over more modules than that described herein. Additionally, other embodiments may consolidate the described functions into fewer modules.

Although various features may have been described with respect to less than all embodiments, this disclosure contemplates that those features may be included on any embodiment. Further, although the embodiments described herein may have omitted some combinations of the various described features, this disclosure contemplates embodiments that include any combination of each described feature. Accordingly, departure in form and detail may be made without departing from the scope and spirit of the present disclosure as described in the appended claims.

What is claimed is:

1. A catheter system comprising:
a catheter including one or more lumens;
an elongate member advanceable through a lumen of the one or more lumens such that a distal end portion of the elongate member extends distally of a distal end of the catheter;
a sensor positioned at a distal end portion of the elongate member and configured to sense a value at a location distal of the distal end of the catheter; and
an aspiration pump in communication with an aspiration lumen of the one or more lumens of the catheter; and
wherein the aspiration pump initiates aspiration in response to the sensor sensing a value that reaches and/or is beyond an aspiration threshold value.

2. The catheter system of claim 1, further comprising:
a controller in communication with the sensor; and
wherein the controller is configured to receive the value from the sensor and compare the value to the aspiration threshold value.

3. The catheter system of claim 1, wherein:
the catheter includes an expandable member having a collapsed configuration and an expanded configuration; and
the expandable member is configured to expand to the expanded configuration in response to the value from the sensor reaching and/or going beyond an expansion threshold value.

4. The catheter system of claim 3, further comprising:
an inflation pump in communication with the expandable member; and
wherein the inflation pump is configured to adjust the expandable member between the collapsed configuration and the expanded configuration in response to the value from the sensor reaching and/or going beyond the expansion threshold value.

5. The catheter system of claim 3, further comprising:
a controller in communication with the sensor; and
wherein the controller is configured to receive the value from the sensor and compare the value to the expansion threshold value.

6. The catheter system of claim 5, wherein the controller is configured to initiate expanding the expandable member to the expanded configuration in response to determining the value reaches and/or is beyond the expansion threshold value.

7. The catheter system of claim 5, wherein the expansion threshold value is the same as the aspiration threshold value.

8. The catheter system of claim 5, wherein the controller is configured to initiate expanding the expandable member to the expanded configuration at a first time and is configured to actuate the aspiration pump at a second time after the first time.

9. The catheter system of claim 1, further comprising:
a filter in communication with the aspiration lumen; and
wherein the filter receives fluid passing through the aspiration lumen in response to actuation of the aspiration pump.

10. The catheter system of claim 1, wherein the value sensed by the sensor includes one or more of an impedance measure of a fluid at the location distal of the distal end of the catheter, a temperature measure of a fluid at the location distal of the distal end of the catheter, and a wavelength measure of a fluid at the location distal of the distal end of the catheter.

11. The catheter system of claim 1, wherein the catheter comprises:
an outer catheter; and
an inner catheter extending within a lumen of the outer catheter; and
wherein the inner catheter comprises the aspiration lumen.

12. The catheter system of claim 1, wherein the catheter comprises:
an outer catheter having an opening through a side wall defining a lumen of the outer catheter; and
an inner catheter extending within the lumen of the outer catheter; and
wherein the inner catheter is movable relative to the outer catheter to selectively cover the opening.

13. A contrast removal system for removing contrast from a vascular system, the system comprising:
a catheter having an expandable member and an aspiration lumen having a distal end at location distal of the expandable member;
a pump in communication with the aspiration lumen;
a sensor positionable at a location distal of the distal end of the aspiration lumen, the sensor configured to sense values in fluid at the location distal of the distal end of the aspiration lumen;
a controller in communication with the pump and the sensor; and
wherein the controller is configured to:
receive values from the sensor;
compare the received values to a threshold value; and
initiate the pump when a value of the received values reaches and/or is beyond the threshold value.

14. The system of claim 13, wherein the controller is configured to initiate expanding the expandable member from a collapsed configuration to an expanded configuration in response to the value reaching and/or going beyond the threshold value.

15. The catheter system of claim 13, wherein the catheter comprises:
an outer catheter; and
an inner catheter extending within a lumen of the outer catheter; and
wherein the inner catheter comprises the aspiration lumen.

16. The catheter system of claim 13, wherein the catheter comprises:
an outer catheter having an opening through a side wall defining a lumen of the outer catheter; and
an inner catheter extending within the lumen of the outer catheter; and
wherein the inner catheter is movable relative to the outer catheter to selectively cover the opening.

17. The system of claim 13, wherein the catheter comprises an extension member and the sensor is located on the extension member.

18. The system of claim 13, further comprising:
an elongate member configured to extend to a location distal of a distal end of the catheter; and
wherein the sensor is located at a distal end portion of the elongate member.

19. A method of removing contrast from a patient's vascular system, the method comprising:
inserting a sensor into a vessel of a patient at a location spaced from and distal of a distal end of an aspiration lumen in a catheter, wherein the catheter comprises:
an expandable member; and
the aspiration lumen, where the distal end of the aspiration lumen is distal of the expandable member;
comparing a value sensed by the sensor at the location spaced from and distal of the distal end of the aspiration lumen to a threshold value;
initiating expansion of the expandable member in response to a determination that the value reaches and/or is beyond the threshold value; and
initiating suction of fluid through the aspiration lumen in response to the determination that the value reaches and/or is beyond the threshold value.

20. The method of claim 19, further comprising:
after initiating suction of fluid, stopping the suction of fluid at a predetermined time after determining that the value reaches and/or is beyond the threshold value again.
